(19) United States
(12) Patent Application Publication
(10) Pub. No.: US 2024/0115836 A1
(43) Pub. Date: Apr. 11, 2024

(54) CATHETER SYSTEM WITH CONTROL MECHANISM

(71) Applicant: EDWARDS LIFESCIENCES CORPORATION, Irvine, CA (US)

(72) Inventor: Morgan House, Newfields, NH (US)

(21) Appl. No.: 18/392,305

(22) Filed: Dec. 21, 2023

Related U.S. Application Data
(63) Continuation of application No. 17/359,450, filed on Jun. 25, 2021, now Pat. No. 11,883,611.
Application No. 17/359,450 is a continuation of application No. 15/956,395, filed on Apr. 18, 2018, now Pat. No. 11,045,627.
(60) Provisional application No. 62/486,956, filed on Apr. 18, 2017.

(51) Int. Cl. A61M 25/01

(52) U.S. Cl. CPC A61M 25/0147; A61M 25/0136; A61M 2025/09116

(57) ABSTRACT

A catheter assembly comprises a catheter, a handle, disposed at a proximal part of the catheter, a pull wire, extending from a distal part of the catheter, proximally through the catheter to the handle, and a control handle assembly, disposed at the handle, and configured to be operated: (i) in a gross-tensioning manner that grossly adjusts tension on the pull wire, and (ii) in a fine-tensioning manner that finely adjusts the tension on the pull wire. Other embodiments are also described.

### CROSS-REFERENCE TO RELATED PATENT APPLICATIONS

This application is a Continuation of U.S. Ser. No. 17/359,450 to House, filed Jun. 25, 2021; which is a Continuation of U.S. Pat. No. 11,045,627 to House, filed Apr. 18, 2018; which is based on and claims priority to Provisional U.S. Patent Application 62/486,956, filed Apr. 18, 2017. Each of the above applications is incorporated by reference herein as if expressly set forth in its respective entirety herein.

### TECHNICAL FIELD

The present teachings generally relate to a delivery catheter with a deflectable distal portion. In one aspect, the delivery catheter can include a control mechanism for precisely controlling the deflecting angle of the distal portion of the delivery catheter.

### BACKGROUND

Deflectable catheters usually feature a tip that can be pulled into a defined curve. This deflection of the catheter tip is independent of the rest of the catheter. Such movement can be achieved by exerting a force biased to one side of the distal portion by using a wire connected to a pull or anchor ring near the tip. The catheter tip can return to its original shape when the force is reduced or removed.

Deflectable catheters have been used in cardiology, peripheral vascular therapies, structural heart therapies, and many other fields that require the catheter tip to make angulated turns or to be fairly accurately positioned in an anatomy. Examples include guiding catheters, implant delivery systems, or EP mapping catheters, and ablation catheters.

The deflectable catheters in the market include uni-directional catheters, bi-directional catheters, 4-way deflectable catheters, and omni-directional catheters. A bi-directional catheter features a tip that can be pulled in two directions (often opposite from each other). This can be achieved by using two pull wires connected to a distal pull ring. A 4-way deflectable catheter can be pulled in 4 directions. The 4-way deflectable catheter often requires four wires connected to a distal pull ring. An omni-directional catheter is a 4-way deflectable catheter that is often remotely controlled by a robotic device to allow the tip to be deflected in any direction. Deflection is achieved by manipulating one or more of the pull wires simultaneously. Robotic catheters can be used for a variety of applications and provide the physician with a greater control and less exposure to radiation.

The deflectability of the catheter tip can be qualified in many ways. A “curve angle” is measured as the angle of the tip movement relative to its straight axis, i.e. the bend angle. The term “bend radius” refers to the inside curvature of the catheter and indicates the minimum radius one can bend a catheter without kinking it. Most deflectable catheters have a curve angle ranging between about 45 and about 180 degrees depending on the application, but can be up to about 270 degrees or in some instances 360 degrees. A “curve diameter” indicates the furthest distance that the catheter moves from its straight axis as it is being deflected. The “reach” measures the displacement of the tip from its central or straight axis.

Deflectable catheters also are categorized as single plane deflection catheters and bi-plane deflection catheters. A single plane deflection catheter deflects within a single plane and includes all uni-directional catheters and most bi-directional catheters. The tip of a bi-plane deflection catheter can deflect along X and Y axis. In other words, it turns side to side and forwards or backwards. Bi-plane deflection catheters include 4-way deflectable catheters.

The deflection of the catheter tip is typically achieved by one or more pull wires via a control mechanism. The most common control mechanism is a simple push-pull mechanism that extends or retracts the pull wire and thereby actuates the deflection of the catheter tip. Thus, the relative linear motion of the push-pull mechanism decides the bend angle and control the planarity of the catheter tip. Although easy to operate, the linear motion of the push/pull mechanism lacks the ability to precisely control the bend angle. In percutaneous applications, the curve angle of a deflectable tip needs to be able to be meticulously adjusted in order for a clinician to find a desired location inside each individual anatomy. Thus, the lack of the ability to be finely adjusted must be improved to allow a clinician to better treat patients.

### SUMMARY

One aspect of the present teachings provides a catheter assembly that comprises a catheter shaft and a control mechanism. The catheter shaft has a deflectable distal portion. The control mechanism is configured to activate a rapid transformation of the deflectable distal portion of the catheter shaft from a linear profile to a curved profile. The control mechanism further comprises a linear actuation mechanism. The linear actuation mechanism converts a rotation motion of the control mechanism to a precise linear motion. The linear motion of the control mechanism is configured to control the bend angle of the deflectable distal portion of the catheter shaft.

Another aspect of the present teachings provides a catheter assembly that comprises a catheter shaft, a pull wire joining the distal end of the catheter shaft, and a control mechanism. The pull wire is configured to deflect a distal portion of the catheter shaft. The control mechanism includes a compression tube mount, and a pull wire mount. A proximal portion of the catheter shaft joins the compression tube mount. A proximal end of the pull wire joins the pull wire mount. A change in distance between the pull wire mount and the compression tube mount results the deflection of the distal portion of the catheter shaft.

Another aspect of the present teachings provides a control mechanism comprising an outer handle shaft, a middle handle shaft. The middle handle shaft is positioned inside an interior lumen of the outer handle shaft. The middle handle shaft has a first position wherein the middle handle shaft engages an interior surface of the outer handle shaft via a thread engagement, and a second position wherein the middle handle moves laterally without the restriction of the thread engagement.

Another aspect of the present teachings provides a control mechanism having a linear actuator. The linear actuator comprises a threaded rotator and a thread follower with pairing threads. The linear actuator is configured to convert the rotational motion of the threads into a relative linear motion of the middle handle and the outer handle shaft.

Another aspect of the present teachings provides a control assembly having the middle handle shaft automatically centers within the outer handle shaft by two centering springs. Each centering spring is placed on each side of the middle handle shaft. When the centering spring is in its relaxed state, the middle handle shaft in centered within the interior lumen of the outer handle shaft.

Another aspect of the present teachings provides the middle handle shaft having a thread engagement mechanism. The thread engagement mechanism comprises a thread follower that engages a thread inside the interior luminal surface of the outer handle shaft. The thread engagement mechanism further comprises a spring. When the spring is compressed, the thread follower engages the thread inside the interior luminal surface of the outer handle shaft. When the spring relaxes, the thread follower disengages from the threads inside the interior luminal surface of the outer handle shaft.

Another aspect of the present teachings provides a catheter assembly that comprises a catheter shaft and a control mechanism. The catheter shaft comprises a first configuration where a distal portion aligns with the longitudinal axis of the catheter shaft, and a second configuration where the distal portion curves away from the longitudinal axis of the catheter shaft.

### BRIEF DESCRIPTION OF THE DRAWINGS

FIG. 1 is a perspective view of an embodiment of the present teachings where a delivery catheter assembly includes a control handle assembly and a catheter shaft with a deflectable distal portion.

FIGS. 2A-2B are perspective views of a distal portion of the catheter shaft, according to one embodiment of the present teaching.

FIG. 2C is a perspective view of a control handle assembly, according to one embodiment of the present teaching.

FIGS. 3A-3D are perspective views of a control handle assembly, according to one embodiment of the present teaching.

FIGS. 4A-4D are perspective views of a delivery catheter assembly including a control handle assembly and a catheter shaft with a deflectable distal portion at various operating stage, according to one embodiment of the present teaching.

FIGS. 5A-5E are perspective views of a control handle assembly at various operating stage, according to one embodiment of the present teaching.

### DETAILED DESCRIPTION OF CERTAIN EMBODIMENTS

Certain specific details are set forth in the following description and figures to provide an understanding of various embodiments of the present teachings. Those of ordinary skill in the relevant art would understand that they can practice other embodiments of the present teachings without one or more of the details described herein. Thus, it is not the intention of the applicant(s) to restrict or in any way limit the scope of the appended claims to such details. While various processes are described with reference to steps and sequences in the following disclosure, the steps and sequences of steps should not be taken as required to practice all embodiments of the present teachings.

As used herein, the term “lumen” means a canal, a duct, or a generally tubular space or cavity in the body of a subject, including a vein, an artery, a blood vessel, a capillary, an intestine, and the like. The term “lumen” can also refer to a tubular space in a catheter, a sheath, a hollow needle, a tube, or the like.

As used herein, the term “proximal” shall mean close to the operator (less into the body) and “distal” shall mean away from the operator (further into the body). In positioning a medical device inside a patient, “distal” refers to the direction relatively away from a catheter insertion location and “proximal” refers to the direction relatively close to the insertion location.

As used herein, the term “wire” can be a strand, a cord, a fiber, a yarn, a filament, a cable, a thread, or the like, and these terms may be used interchangeably.

As used herein, the term “sheath” may also be described as a “catheter” and, thus, these terms can be used interchangeably.

Unless otherwise specified, all numbers expressing quantities, measurements, and other properties or parameters used in the specification and claims are to be understood as being modified in all instances by the term “about.” Accordingly, unless otherwise indicated, it should be understood that the numerical parameters set forth in the following specification and appended claims are approximations. At the very least and not as an attempt to limit the application of the doctrine of equivalents to the scope of the claims, numerical parameters should be read in light of the number of reported significant digits and the application of ordinary rounding techniques.

The present teachings relate to an implant delivery catheter with a deflectable catheter tip, an ablation catheter with a deflectable tip or any therapy catheter requiring actuation at the distal section or tip. In some embodiments, the delivery catheter includes a control mechanism. In some embodiments, the control mechanism is a precision control mechanism. In certain embodiments, the control mechanism allows a clinician to control, sometimes accurately, the bend angle, reach and curve diameter of the deflectable tip.

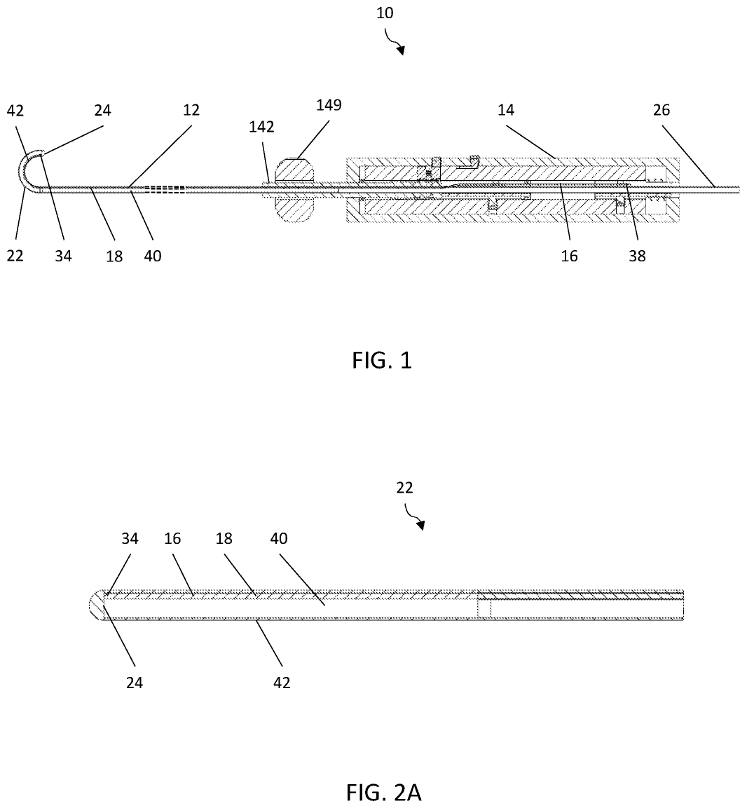

Referring now to FIG. 1, according to one embodiment of the present teachings, the delivery catheter assembly (10) includes a catheter shaft (12) having a distal portion (22), where the distal portion of the catheter shaft (12) is deflectable, and a control handle assembly (14) disposed at the proximal portion (26) of the catheter shaft (12). In some embodiments, the control handle assembly (14) is connected with the proximal portion (26) of the catheter shaft (12). In some embodiments, the control handle assembly (14) includes a pull wire (16). In some embodiments, the pull wire (16) extends along the longitudinal axis of the catheter shaft (12). In some embodiments, the pull wire (16) connects its distal end (34) to the distal end (24) of the catheter shaft (12) and connects the control handle assembly (14) at the proximal end (38). In some embodiments, when a clinician applies tension on the proximal end (38) of a pull wire (16), such tension is transferred to the distal end (34) of the pull wire (16) and through the distal end (34) of the pull wire (16) that is connected with the distal end (24) of the catheter shaft (12). As a result, the catheter shaft (12) articulates in a single direction. In some embodiments, the pull wire (16) is positioned inside a peripheral lumen (18) extending along the catheter shaft (12). In some embodiments, the direction of the articulation is defined by the positions of the pull wire (16), or the peripheral lumen (18) that housing the pull wire (16), in reference to the center axis of the catheter shaft (12).

According to some embodiments, a catheter shaft (12) of the present teachings include a longitudinal axis that runs from its proximal end (28) to its distal end (24). In some embodiments, the catheter shaft (12) includes a central longitudinal lumen (40). In some embodiments, the central longitudinal lumen (40) allows an implant, including an RF wire or other devices, to slide through. In some embodiments, the catheter shaft (12) includes an elongated and generally flexible portion (42) and an articulable distal portion (22). In some embodiments, the articulable distal portion (22) is configured to bend, curve, or otherwise change its shape and position. In some embodiments, the articulation of the distal portion (22) is triggered by the control handle assembly (14).

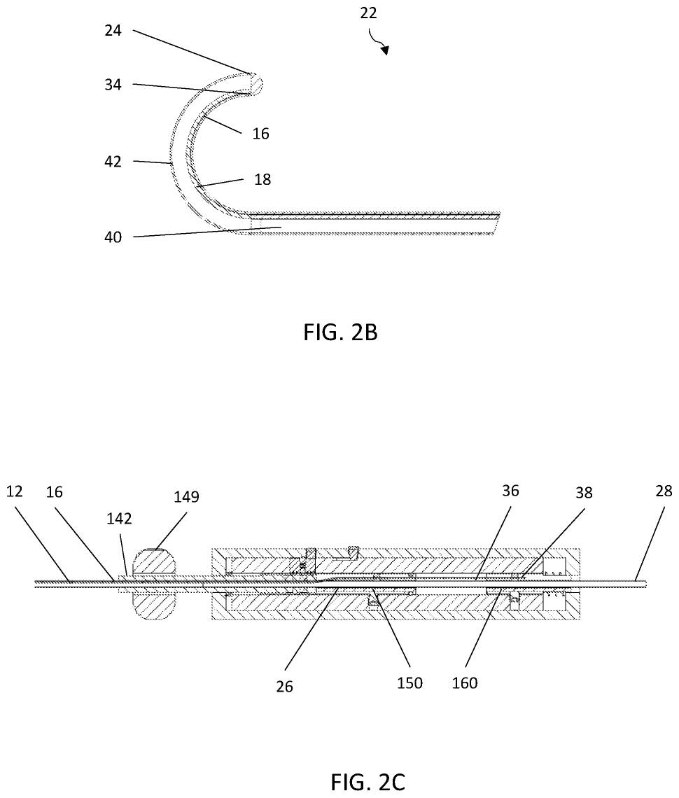

FIGS. 2A-2B illustrate one embodiment of the articulable distal portion (22). As shown in FIG. 2A, the pull wire (16) runs inside a peripheral lumen (18) exterior to the central longitudinal lumen (40) of the catheter shaft (12) along the length of the articulable distal portion (22) of the catheter shaft (12). The pull wire (16) attaches to the distal end (24) of the catheter shaft (12) by an attaching means. In some embodiments, the attaching means is a ring at the distal tip of the catheter shaft (12) to which the pull wire (16) is soldered. In some embodiments, the attaching means includes the pull wire (16) being embedded in the composite plastic or polymer material at the tip of the catheter shaft (12). In some embodiments, the pull wire (16) embedded in a composite of plastic or polymer are coiled at the attaching location, for example, to provide a strong attachment and to prevent the pull wire (16) from being pulled out of the composite material when tension is applied. As the tension is applied to the pull wire (16), the distal portion (22) of the catheter shaft (12) articulates in a single direction as shown in FIG. 2B.

According to some embodiments of the present teachings, both the generally flexible portion (42) and an articulable distal portion (22) of the catheter shaft (12) have a bending stiffness that allows the catheter (12) to be trans-luminally positioned through a tortuous path into the heart. According to some embodiments of the present teachings, the bending stiffness of the articulable distal portion (22) of the delivery catheter (12) is substantially less than the generally flexible portion (42) of the catheter shaft (12). In some embodiments, the catheter shaft (12) has sufficient column strength to remain substantially un-deflected when the pull wire (16) is tensioned. In some embodiments, the articulable distal portion (22) of the delivery catheter (12) is sufficiently flexible for deflection into a curvature.

In some embodiments, a middle portion (35) of the pull wire (16) is disposed within a peripheral lumen (18) unattached to the catheter shaft (12). The proximal end (38) of the pull wire (16) can be attached to the control handle assembly (14) as shown in FIG. 1. A proximal portion (26) of the catheter shaft (12) can be connected to the control handle assembly (14). According to one embodiment of the present teachings, the control handle assembly (14) is configured to apply an axial motion to manipulate the pull wire (16). In some embodiments, the manipulation of the pull wire (16) results in deflecting the distal portion (22) of the catheter shaft (12).

FIG. 2C illustrates the proximal end portion (36) of the pull wire (16) and the catheter shaft (12). As shown in FIG. 2C, the proximal end (38) of the pull wire (16) is fixed to a pull wire mount (160). The proximal portion (26) of the catheter shaft (12) is fixed to a compression tube mount (150). The proximal end (28) of the catheter shaft (12) further extends proximally beyond the control handle assembly (14). The relative movement of the compression tube mount (150) and pull wire mount (160) will be translated to a relative motion between the catheter shaft (12) and pull wire (16), and thereby deflects the distal portion (22) of the catheter shaft (12).

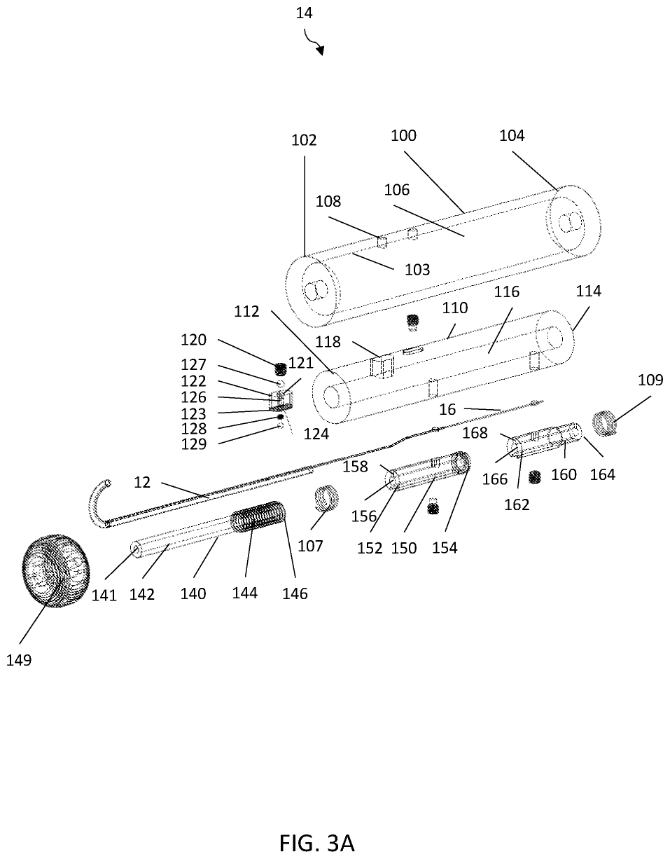

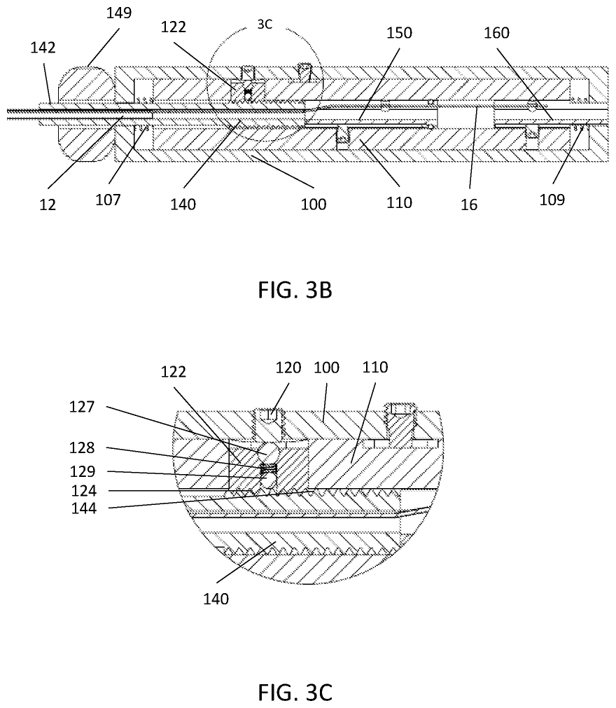

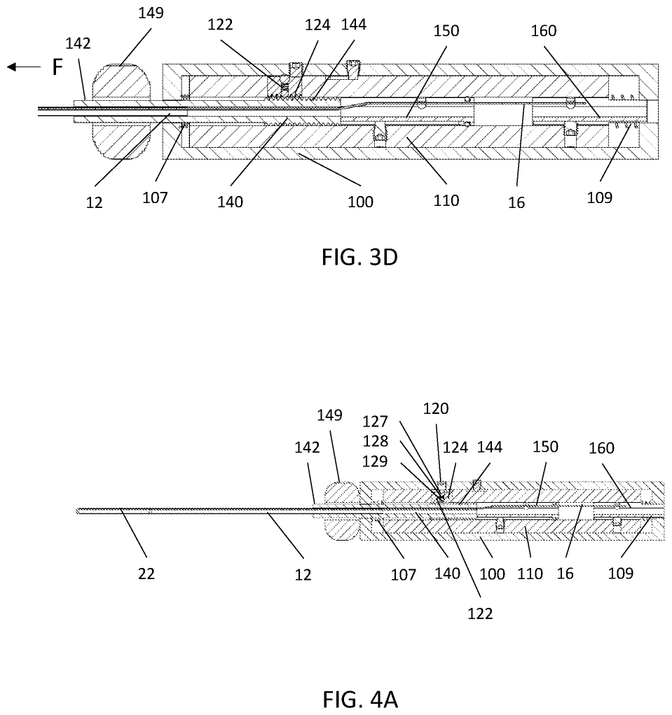

FIG. 3A illustrates an exploded view of the control handle assembly (14). FIG. 3B illustrates an assembled view of the control handle assembly (14). Now referring FIG. 3A, the control handle assembly (14) includes an elongated hollow outer handle shaft (100). In some embodiments, a hollow middle handle shaft (110) is disposed within the elongated hollow outer handle shaft (100). The distal end (102) of the outer handle shaft (100) is sized to allow a threaded rotator (140) to extend through, while block the middle handle shaft (110) in the outer handle shaft (100) from sliding proximally out. The proximal end (104) of the outer handle shaft (100) is sized to allow a catheter shaft (12) to extend through, while also blocking the middle handle shaft (110) from sliding distally out. The outer handle shaft (100) further has a center lumen (106) and a through hole (108) that extends from the center lumen (106) radially to the exterior surface (101) of the outer handle shaft (100). In some embodiments, the size of the through hole (108) is configured to house a set screw (120). In some embodiments, the location of the through hole (108) could vary along the outer handle shaft (100), so long as it centers over the thread follower (122) when the middle handle shaft (110) centers within the outer handle shaft (100). In some embodiments, a longitudinal slot is designed to allow a first ball bearing (127) to slide within. According to this embodiment, the longitudinal slot is placed on the interior surface (103) of the center lumen (106) starts near the through hole (108) and extends distally to the distal end (102) of the outer handle shaft (100).

FIG. 3A further illustrates an elongated middle handle shaft (110) having a center lumen (116) extending from its distal end (112) to its proximal end (114). The middle handle shaft (110) further includes a thread follower retainer slot (118) Similar to the through hole (108), the placement of the thread follower retainer slot (118) could vary so long as it allows the thread follower (122) centers under the set screw (120) when the middle handle shaft (110) centers within the outer handle shaft (100). The thread follower retainer slot (118) is designed to receive a thread follower (122). The center lumen (116) of the middle handle shaft (110) is sized to receive a threaded rotator (140), a compression tube mount (150), and a pull wire mount (160).

Continue referring to FIG. 3A, the thread follower (122) has a top side (121) and a bottom side (123). The bottom side (123) of the thread follower (122) has threads (124) configured to match the threads (144) of the threaded rotator (140). The thread follower (122) further includes a hole (126) for holding a first ball bearing (127) followed by a spring (128) in the middle and a second ball bearing (129). In some embodiments, the ball bearings (127, 129) and the spring (128) are sized to be housed inside the hole (126), while the spring (128) is trapped in between the two ball bearings (127, 129) as shown in FIG. 3B. In some embodiments, when the middle handle shaft (110) is centered inside the outer handle shaft (100), the first ball bearing (127) is then resume its position and engages under the set screw (120). The set screw (120) then compresses the first ball bearing (127) downward. The first ball bearing (127) in turn compresses the spring (128) and thereby also pushes the thread follower (122) downward. In another embodiment, as the first ball bearing (127) moves away from its position under the set screw (120), the spring (128) relaxes and thereby allows the thread follower (122) moving upward.

Continue referring to FIG. 3A, a threaded rotator (140), a compression tube mount (150), and a pull wire mount (160) are placed inside the center lumen (116) of the middle handle shaft (110). In some embodiments, the threaded rotator (140), the compression tube mount (150), and the pull wire mount (160) are arranged from the distal end (112) to the proximal end (114) of the middle handle shaft (110). In some embodiments, the threaded rotator (140) has an elongated hollow body with a threaded proximal portion (144) and an unthreaded distal portion (142). As shown in FIG. 3A, the distal portion (142) extends from inside the middle handle shaft (110) and outer handle shaft (100) distally to the outside. In some embodiments, a steering knob (149) attaches to the distal portion (142) of the threaded rotator (140) that is outside of the outer/middle handle shafts (100, 110). In some embodiments, the proximal end (146) of the threaded rotator (140) is disposed next to a compression tube mount (150). In some embodiments, the threaded rotator (140) has a longitudinal lumen (141) configured to receive a catheter shaft (12).

FIG. 3A illustrates a compression tube mount (150) is positioned proximal to the threaded rotator (140). In some embodiments, the compression tube mount (150) has an elongated hollow body with an axial lumen (156) extending from one end (152) to the other end (154). The distal end (152) of the compression tube mount (150) butts against the proximal end (146) of the threaded rotator (140). In some embodiments, the threaded rotator (140) and the compression tube (150) are configured to move laterally together, while the threaded rotator (140) rotates independently from the compression tube mount (150). According to one embodiment of the present teaching, as the threaded rotator (140) rotates against the thread follower (122), the threaded rotator (140) also moves laterally, and the compression tube mount (150) moves laterally along with the threaded rotator (140). In some embodiments, such threaded rotator (140) and compression tube (150) assembly configuration can be achieved by many means known to those skilled in the art. For example, a first end cap could be used to stop proximal motion of the threaded proximal portion (144) of the threaded rotator (140), and a second end cap could be used to stop distal motion of the proximal end (154) of the compression tube mount (150).

Continue referring to FIG. 3A, the compression tube mount (150) has a key slot (158) extending from its axial lumen (156) radially away to the exterior surface. The key slot (158) is configured to receive a key on the catheter shaft (12). Through this key slot-key assembly, the catheter shaft (12) is bonded to the compression tube mount (150). Such bonding assembly prevents the catheter shaft (12) from rotating and moving laterally relative to the compression tube mount (150). In some embodiments, key slot-key assembly allows the lateral movement of the threaded rotator (140) and compression tube (150) assembly being translated to the catheter shaft (12).

FIG. 3A further illustrates a pull wire mount (160) placed proximally to the compression tube mount (150) Similar to the compression tube mount (150), the pull wire mount (160) also has an elongated hollow body with an axial lumen (166) extending from one end (162) to the other end (164). The catheter shaft (12) is slidably disposed within the axial lumen (166) of the pull wire mount (160). As shown in FIG. 3A, the pull wire mount (160) also has a key slot (168) extending from its axial lumen (166) radially away to the exterior surface. The key slot (168) is configured to receive the proximal end of the pull wire (16). In some embodiments, the proximal end of the pull wire (16) is bonded to the key slot (168) of the pull wire mount (160). According to some embodiment, the pull wire mount (160) is fixed to the middle handle shaft (110) so that the pull wire mount (160) is prevented from rotating and moving laterally relative to the middle handle shaft (110).

Now referring to FIG. 3B, the catheter shaft (12) extends proximally through the axial lumen (141) of the threaded rotator (140), the axial lumen (156) the compression tube mount (150), the axial lumen (166) of the pull wire mount (160), and then further extends proximally beyond the outer handle shaft (100), thus outside of the control handle assembly (14). According to one embodiment of the present teachings, the proximal end of the shaft is used to insert a medical implant, or as the entrance for an RF wire.

Continue referring FIG. 3B, as described above, the catheter shaft (12) bonds to the compression tube mount (150) with the key on the catheter shaft (12) disposed within the key slot (158) of the compression tube mount (150). The proximal end of the pull wire (16) bonds to the pull wire mount (160). The threaded rotator (140) joins the compression tube mount (150), together they are position distal to the pull wire mount (160). As shown in FIG. 3B, the pull wire mount (160) is proximal to the compression tube mount (150), and the threaded rotator (140) is distal to the compression tube mount (150) with the threaded proximal portion (144) next to the compression tube mount (150) and the unthreaded distal portion (142) outside of the outer handle shaft (100).

Continue referring to FIG. 3B, the pull wire mount (160), the compression tube mount (150) and the threaded rotator (140) assembly are placed inside the center lumen (116) of the middle handle shaft (110). As shown in FIG. 3B, the pull wire mount (160) is fixed to a location inside the middle handle shaft (110). The compression tube mount (150) joins to the threaded rotator (140) forming an assembly which moves distally or proximally. Such laterally movement is translated to the catheter shaft (12) through the bonding between the catheter shaft (12) and the compression tube mount (150). The movement of the compression tube mount (150) and the threaded proximal portion (144) of threaded rotator (140) assembly is limited inside the middle handle shaft (110). Although the threaded rotator (140) and compression tube assembly is configured to slide within the center lumen of the middle handle shaft (110), and thereby pulling or pushing the catheter shaft distally or proximally. The unthreaded distal portion (142) of the threaded rotator (140) extends distally outside of the middle handle shaft (110). A steering knob (149) attaches to the distal portion (142) of the threaded rotator (140). The steering knob (149) can be used by a clinical control to push, pull, and rotate the thread rotator.

Further referring to FIG. 3C, the thread follower (122) is placed inside the thread follower retainer slot (118) on the middle handle shaft (110) with the bottom threads (124) facing the threads (144) of the threaded rotator (140). A set screw (120) is place inside the through hole (108) on the outer handle shaft (100). As illustrated in FIG. 3C, the set screw (120) slightly protrudes beyond the interior slotted surface of the outer handle shaft (100). FIG. 3C illustrates the thread follower (122) centers under the set screw (120). As shown in FIG. 3C, the set screw (120) presses on the first ball bearing (127), the first ball bearing (127) then compresses the spring (128), the spring (128) then forces the second bear bearing (129) against the bottom of the hole. The thread follower (122) then engages toward the threaded rotator (140). Threads (124) thereby engages the threads (144) of the threaded rotator (140).

Referring again to FIG. 3B, the middle handle shaft (110), with all the assembly inside its center lumen (116), is disposed inside the center lumen (106) of the outer handle shaft (100). Two springs (107, 109), including a proximal spring (109) and a distal spring (107), are placed at each end of the middle handle shaft (110). These two springs (107, 109), at their relaxed state, force the middle handle shaft (110) inside the outer handle shaft (100) to its first position where the thread follower (122) centers under the set screw (120) thereby allowing the threads (124) of the thread follower (122) engages the threads (144) of the threaded rotator (140) as described above and illustrated in FIG. 3C. When the middle handle shaft (110) is being pulled distally with a force “F”, the distal spring is compressed, the first ball bearing (127) is freed from the set screw (120). As the first ball bearing enters the longitudinal slot (105) on the interior surface (103) of the outer handle shaft (100), the spring (108) relaxed, and the thread follower (122) then moves away from the thread rotator (140). Engagement between threads (124, 144) is then released, and the middle handle shaft (110) is now in its second position where the thread rotator (140) and the compression tube mount (150) assembly moves distally and proximally with lateral force without the constriction of the threads (124, 144) engagement, as illustrated in FIG. 3D. Once the pulling force “F” is released, the proximal spring recovers and pushes the middle handle assembly back to its first position as illustrated in FIG. 3B.

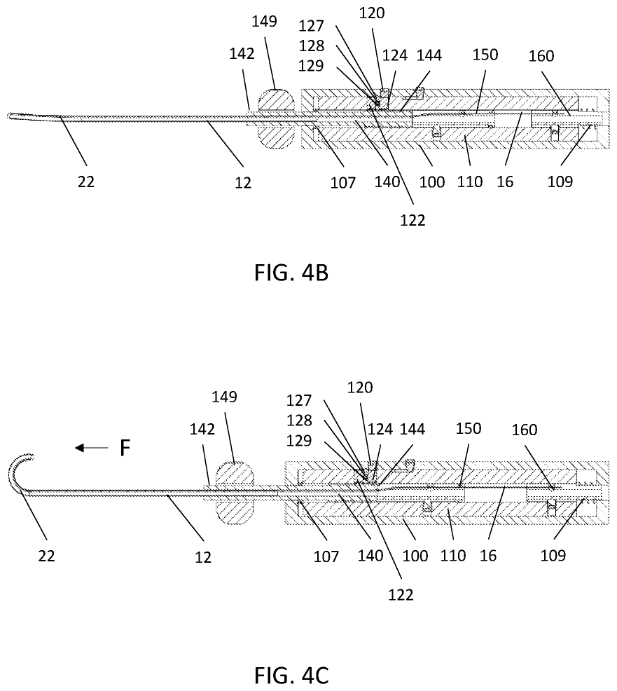

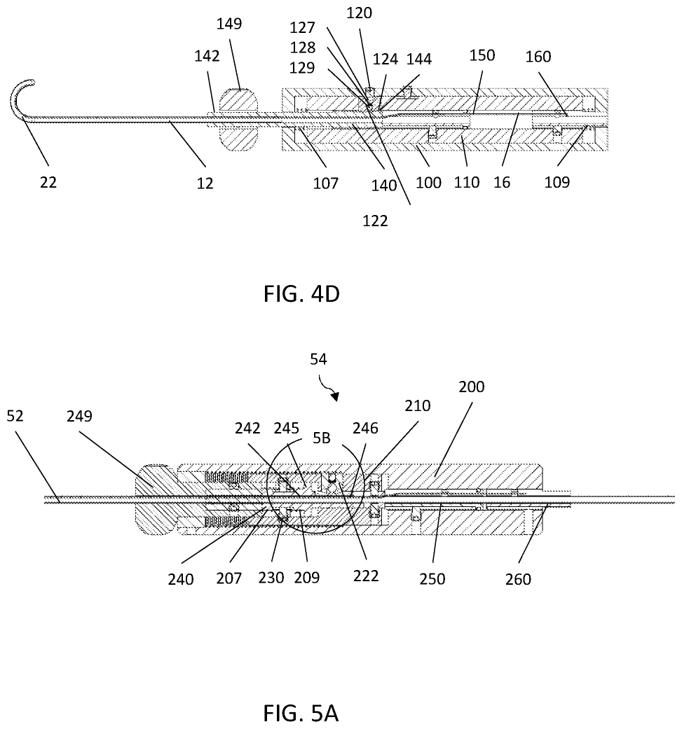

FIGS. 4A-4E illustrate the moving mechanism of the control handle assembly (14) in FIGS. 3A-3C. As shown in FIG. 4A, the middle handle shaft (110) is at its first position within the outer handle shaft (100) and the springs (107, 109) are in their relaxed state. In operation, the clinician holds the outer handle shaft (100) steady, and pulling steering knob (149) distally. As illustrated in FIG. 4B, the first ball bearing (127) is released from the set screw (120), the spring (128) recovers to its relaxed state, the threads (124, 144) disengage from each other, the thread follower (122) floats, and the middle handle shaft (110) is now in its second position. While keep holding the outer handle shaft (100) steady, the clinician can continue pull the steering knob (149) distally, the threaded rotator (140) and compression tube mount (150) assembly slide laterally inside the middle handle shaft (110), as illustrated in FIG. 4C, and carrying catheter shaft (12) with it. The distal portion (22) of the catheter shaft (12) deflects as the distance between the pull wire mount (160) and compression tube mount (150) changes. Thus such push-pull of the steering knob (149) leads to a gross displacement of the distal portion (22) of the catheter shaft (12).

As the clinician releases the outer handle shaft (100), the springs (107, 109) recover and the middle handle shaft (110) resumes its first position within the outer handle shaft. The set screw (120) employs the first ball bearing (127), and the thread follower (122) engages the threaded rotator (140) as illustrated FIG. 4D. According to some embodiments, the threads engagement functions as a linear actuator and converts the rotational motion of the threads into a linear motion. At this point, the clinician rotates the threaded rotator (140), and such rotation motion is translated into a lateral movement by the threads (124, 144) engagement, thereby allows the threaded rotator (140) and compression tube mount (150) assembly move laterally relative to the pull wire mount (160), as shown in FIG. 4E. Thus, such thread rotation-to-linear actuation leads to a precision displacement of the distal portion (22) of the catheter shaft (12).

In one embodiment of the present teaching, as the threaded rotator (140) rotates 180 degrees, the distal end (24) of the catheter shaft (12) displaces 0.5 mm to 5 mm. In some embodiments, the precision displacement of the distal portion (22) of the catheter shaft (12) depends on the step angle and pitch of the threads (124, 144) assembly. According to some embodiments, the thread (144) could have multiple starts, multiple pitches.

One skilled in the art should understand that, according to some embodiments, the design principle of the present teachings includes a quick linear motion by the control handle assembly to allow a gross displacement of the deflectable distal portion of the catheter shaft, and an automatic engagement of the thread mechanism allowing a rotation-to-linear actuation imparts a precision displacement of the deflectable distal portion of the catheter shaft. The exemplary embodiments shown in FIGS. 3A-3D incorporate such principle. The exemplary embodiments shown in FIGS. 4A-4D explains the working mechanism of such principle. One skilled in the art should understand that other design examples could also be incorporate to embody such principle.

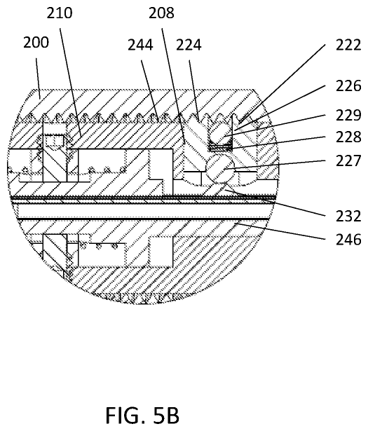

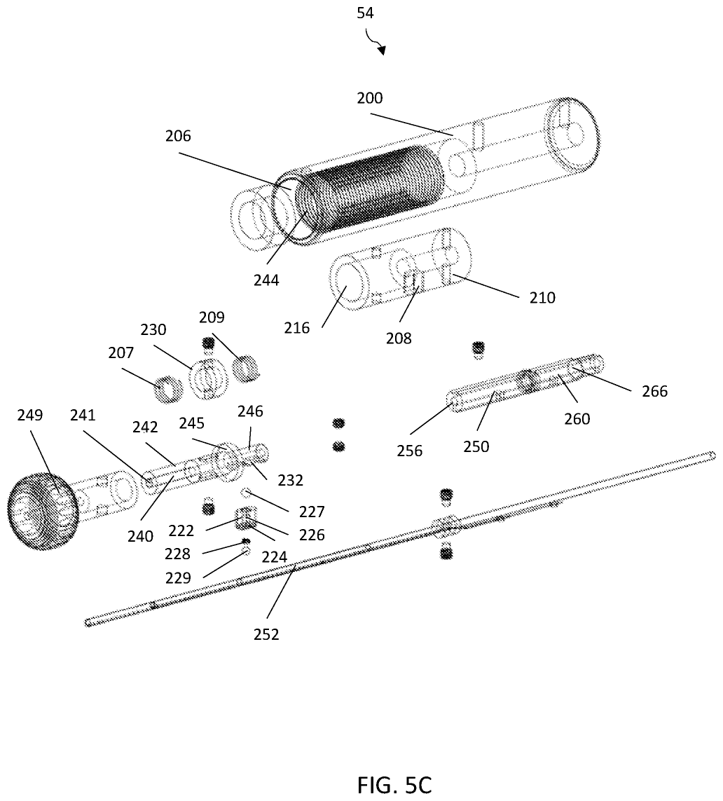

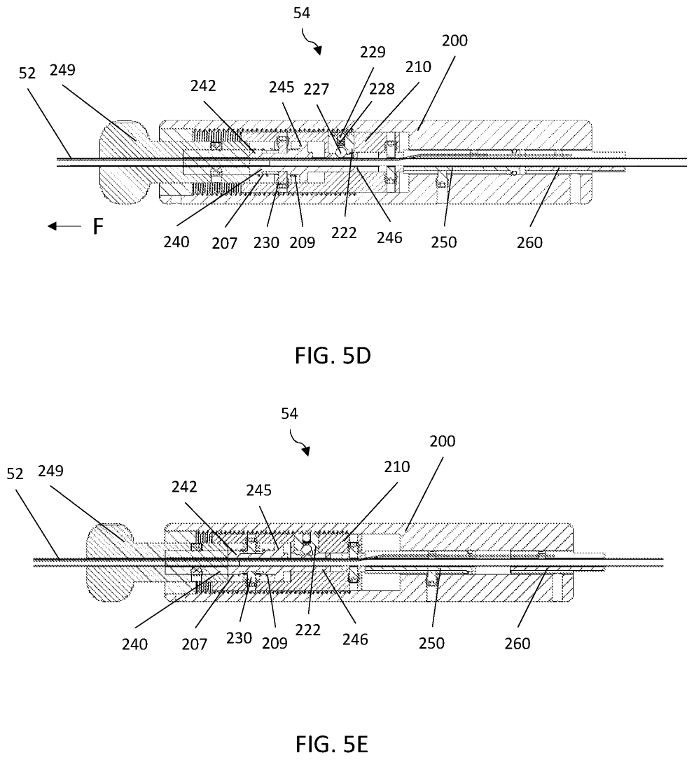

FIGS. 5A-5C further illustrate another embodiment of present teaching. In the most part, this exemplary embodiment is similar to the exemplary embodiment described above. For example, a catheter shaft (52) extends through the center lumen (241) of a rotating cam (240), the center lumen (256) of the compression tube mount (250), and the center lumen (266) of the pull wire mount (260). The catheter shaft (52) extends further proximally outside of the control handle assembly (54). The pull wire mount (260) is proximal to the compression tube mount (250), and the rotator cam (240) is distal to the compression tube mount (250) Similar to described above, catheter shaft (52) fixes to the compression tube mount (250) through a key slot-key assembly, preventing the catheter shaft (52) from rotating and moving laterally relative to the compression tube mount (250). The proximal end of the pull wire also fixes to the pull wire mount (260).

Unlike what has been described above, in this exemplary embodiment, as shown in FIG. 5C, the rotating cam (240) has an enlarged distal portion (242) and a smaller proximal portion (246) with a flange (245) dividing the two portions. A distal knob (249) joins the distal portion (242) of the rotating cam (240). The distal knob (249) also has a center lumen allowing the catheter shaft (52) to extend through. A centering bushing (230) also rides over the distal portion of the rotating cam (240) with two centering springs (207, 209) at each end of the centering bushing (230). As shown in FIG. 5B, the distal knob (249) is distal to the distal spring (207), and the distal spring (207) is distal to the centering bushing (230), and the centering bushing (230) is distal to the proximal spring (209). According to one embodiment, the centering bushing (230) fixes to the distal portion (242) of the rotating cam (240) in such way that the centering bushing (230) is prevented from rotating relative to the rotating cam (240), while allowed to move longitudinally relative to the rotating cam (240).

Further referring to FIG. 5B, unlike exemplary embodiment described in reference to FIGS. 3A-3D, the rotating cam (240) has no threads. Instead, a thread follower (222) positions over a ridge (232) on proximal portion (246) of the rotating cam (240) Similar to what has been described in reference to FIG. 3B, a hole (226) housing a second ball bearing (229) inside and a spring (228) trapped in between of a first ball bearing (227) and the second ball bearing (229). The thread follower (222) also has a thread facing radially away from the rotating cam (240). The threads (224) on the thread follower (222) is configured to engage the threads (244) on the interior surface of the outer handle shaft (200) as described below.

FIG. 5C, further illustrates a middle handle shaft (210) has a center lumen (216) configured to house the rotating cam (240). The middle handle shaft (210) has a through hole (208) extends from its center lumen to its exterior surface, configured to house the thread follower (222). As shown in FIG. 5A, the middle handle shaft (210) joins the compression tube mount (250) at its proximal portion. According to one embodiment of the present teaching, the compression tube mount (250) joins the middle handle shaft (210) in such way that allows the middle handle shaft (210) rotate independently from the compression tube mount (250), while prevent the middle handle shaft (210) from move laterally relative to the compression tube mount (250).

Further referring to FIG. 5A, the outer handle shaft (200) has a center lumen (206) configured to house the pull wire mount (260), the compression tube mount (250) and the middle handle shaft (210) carrying rotating cam (240) and the distal knob (249). The pull wire mount fixes to the outer handle shaft (200), and the distal knob (249) extends distally outside of the center lumen (206) of the distal knob (249).

FIG. 5A illustrates one embodiment of the present teaching where the middle handle shaft (210) is in its first position. As shown in the figure, centering springs (207, 209) are relaxed, the first ball bearing (227) centers over the ridge (232) on the proximal portion (246) of the rotating cam (240). As such, the first ball bearing compresses the spring (228) and the second ball bearing (229), the thread follower (222) thereby forced toward the interior surface of the outer handle shaft (200). Threads (224, 244) then engage each other. At this point, by holding knob (249) steady, a clinician can then rotates the outer handle shaft (200). Since the rotating cam (240) joins the middle handle shaft (210), and the middle handle shaft (210) joins the compression tube mount (250), the threads (224, 244) rotation allows the middle handle shaft (210) and compression tube mount (250) assembly move laterally relative to the pull wire mount (260). Thus, such thread rotation-to-linear actuation leads to a precision displacement of the distal portion (52) of the catheter shaft (52).

FIG. 5D illustrates one embodiment of the present teaching where the middle handle shaft (210) is in its second position. As shown in the figure, as the knob (249) being pulled distally, the proximal spring (209) compresses, and the rotating cam (240) being pulled distally. The ridge (232) on the proximal portion (246) of the rotating cam (240) moves distally relative to the first ball bearing (227). FIG. 5D shows that first ball bearing free from the ridge (232), the spring (228) in between the two ball bearing (227, 229) relaxes. Such relaxation carries the thread follower (222) moving away from the interior thread of the outer handle shaft (200). Threads (224, 244) then disengage each other. At this point, by holding outer handle shaft (200) steady, a clinician call can push the knob (249) distally, thereby changing the distance between the pull wire mount (260) and compression tube mount (250) and leading the distal portion (52) of the catheter shaft (52) deflect. Thus such push-pull of the knob (249) leads to a gross displacement of the distal portion (52) of the catheter shaft (52).

As the clinician releases the outer handle shaft (100), the springs (107, 109) recover and the middle handle shaft (110) resumes its first position within the outer handle shaft (12). The set screw (120) employs the first ball bearing (127), and the thread follower (122) engages the threaded rotator (140) as illustrated FIG. 4D. According to some embodiments, the threads engagement functions as a linear actuator and converts the rotational motion of the threads into a linear motion. At this point, the clinician rotates the threaded rotator (140), and such rotation motion is translated into a lateral movement by the threads (124, 144) engagement, thereby allows the threaded rotator (140) and compression tube mount (150) assembly move laterally relative to the pull wire mount (160), as shown in FIG. 4E. Thus, such thread rotation-to-linear actuation leads to a precision displacement of the distal portion (22) of the catheter shaft (12).

As the clinician releases the force applied to the knob (249), the spring (209) recovers and the middle handle shaft (210) resumes its first position within the outer handle shaft (200). The thread follower (222) repositions over a ridge (232) on proximal portion (246) of the rotating cam (240). The spring (228) compressed and the threads (224, 244) engage each other as illustrated FIG. 5D. According to some embodiments, the threads engagement functions as a linear actuator and converts the rotational motion of the threads into a linear motion. At this point, the clinician rotates the rotating cam (240), and such rotation motion is translated into a lateral movement by the threads (224, 244) engagement, thereby allows the compression tube mount (250) move laterally relative to the pull wire mount (260). Thus, such thread rotation-to-linear actuation leads to a precision displacement of the distal portion of the catheter shaft (52).

According to some embodiments, although exemplary embodiments have been described above in order to explain the present invention, one skilled in the art should understand, design details could be replaced to achieve the same function. For example, set screw (120) described in reference to FIGS. 3A-3D could be place with a ridge design. Proximal and distal springs (107, 109, 207, and 209) could have different size, shape, and recover force. Such design variation should be considered as within the scope of present invention.

Exemplary embodiment shown in FIG. 5A-5D also accomplishes the above described design principle of the present teaching. That is, a quick linear motion by the control handle assembly to allow a gross displacement of the deflectable distal portion of the catheter shaft, and an automatic engagement of the thread mechanism allowing a rotation-to-linear actuation imparts a precision displacement of the deflectable distal portion of the catheter shaft. Thus, those skilled in the art should understand that the exemplary embodiments described above could be modified in various execution while still achieve the same design principle.

Various embodiments have been illustrated and described herein by way of examples, and one of ordinary skill in the art would recognize that variations can be made without departing from the spirit and scope of the present teachings. The present teachings are capable of other embodiments or of being practiced or carried out in various other ways. Also, it is to be understood that the phraseology and terminology employed herein is for the purpose of description and should not be regarded as limiting.

Unless otherwise defined, all technical and scientific terms used herein have the same meaning as commonly understood by one of ordinary skill in the art to which this present teachings belong. Methods and materials similar or equivalent to those described herein can be used in the practice or testing of the present teachings. In case of conflict, the patent specification, including definitions, will control. In addition, the materials, methods, and examples are illustrative only and not intended to be limiting.

1. An apparatus comprising a catheter assembly that comprises:
a catheter;
a handle, disposed at a proximal part of the catheter;
a pull wire, extending from a distal part of the catheter, proximally through the catheter to the handle; and
a control handle assembly, disposed at the handle, and configured to be operated:
in a gross-tensioning manner that grossly adjusts tension on the pull wire, and
in a fine-tensioning manner that finely adjusts the tension on the pull wire.

2. The apparatus according to claim 1, wherein the pull wire is coupled to the catheter such that adjusting the tension in the pull wire adjusts a curvature of the distal part of the catheter.

3. The apparatus according to claim 1, wherein the control handle assembly comprises a knob that is mounted such that the gross-tensioning manner includes sliding of the knob axially with respect to the handle without rotation of the knob.

4. The apparatus according to claim 3, wherein the knob is collinear with the handle.

5. The apparatus according to claim 3, wherein the mounting of the knob is such that:
sliding of the knob axially in a first axial direction with respect to the handle without rotation of the knob grossly increases the tension in the pull wire; and
sliding of the knob axially in a second axial direction with respect to the handle without rotation of the knob grossly reduces the tension in the pull wire, the second axial direction being opposite to the first axial direction.

6. The apparatus according to claim 5, wherein the mounting of the knob is such that sliding of the knob axially in the first axial direction with respect to the handle comprises sliding of the knob distally away from the handle in the first axial direction.

7. The apparatus according to claim 3, wherein the mounting of the knob is such that the fine-tensioning manner includes rotation of the knob with respect to the handle.

8. The apparatus according to claim 7, wherein the mounting of the knob is such that:
rotation of the knob in a first rotational direction with respect to the handle finely increases the tension in the pull wire, and
rotation of the knob in a second rotational direction with respect to the handle finely reduces the tension in the pull wire, the second rotational direction being opposite to the first rotational direction.

9. The apparatus according to claim 1, wherein the control handle assembly comprises (i) a knob, and (ii) a threaded mechanism that provides operative coupling between the knob and the handle, and is configured to be operated:
in the fine-tensioning manner by the threaded mechanism functioning as a linear actuator, and
in the gross-tensioning manner by the threaded mechanism not functioning as a linear actuator.

10. The apparatus according to claim 9, wherein the threaded mechanism is configured such that operation of the control handle assembly in the gross-tensioning manner includes transient disengagement of the threaded mechanism.

11. An apparatus comprising a catheter assembly that comprises:
a catheter;
a handle, disposed at a proximal part of the catheter; and
a knob, disposed at the handle, operatively coupled to a distal part of the catheter, and configured to:
grossly deflect the distal part of the catheter responsively to being moved in an axial, non-rotational manner, and
finely deflect the distal part of the catheter responsively to being rotated.

12. The apparatus according to claim 11, wherein the catheter assembly comprises a wire that is coupled to the catheter, the knob being configured to:
grossly deflect the distal part of the catheter by grossly adjusting tension in the wire, and
finely deflect the distal part of the catheter by finely adjusting the tension in the wire.

13. The apparatus according to claim 11, wherein the knob is collinear with the handle.

14. The apparatus according to claim 11, wherein the catheter assembly comprises a threaded mechanism at the handle that provides operative coupling between the knob and the handle, wherein the knob is configured to:
grossly deflect the distal part of the catheter by the threaded mechanism functioning as a linear actuator, and
finely deflect the distal part of the catheter by the threaded mechanism not functioning as a linear actuator.

15. The apparatus according to claim 14, wherein the threaded mechanism is configured to facilitate movement of the knob in the axial, non-rotational manner via transient disengagement of the threaded mechanism.

16. An apparatus comprising a catheter assembly that comprises:
a catheter;
a handle, disposed at a proximal part of the catheter;
a pull wire, extending from a distal part of the catheter, proximally through the catheter to the handle; and
a control handle assembly, disposed at the handle, and configured to be operated:
in a gross-tensioning manner that grossly reshapes the distal part of the catheter, and
in a fine-tensioning manner that finely reshapes the distal part of the catheter.

17. The apparatus according to claim 16, wherein the pull wire is coupled to the catheter, and the control handle assembly is configured to:
grossly reshape the distal part of the catheter by grossly adjusting tension in the pull wire, and
finely reshape the distal part of the catheter by finely adjusting the tension in the pull wire.

18. The apparatus according to claim 16, wherein the control handle assembly comprises a knob that is mounted such that the gross-tensioning manner includes sliding of the knob axially with respect to the handle without rotation of the knob.

19. The apparatus according to claim 18, wherein the mounting of the knob is such that the fine-tensioning manner includes rotation of the knob with respect to the handle.

20. The apparatus according to claim 18, wherein the control handle assembly comprises a threaded mechanism that provides operative coupling between the knob and the handle, and is configured to be operated:
in the fine-tensioning manner by the threaded mechanism functioning as a linear actuator, and
in the gross-tensioning manner by the threaded mechanism not functioning as a linear actuator.

21. The apparatus according to claim 20, wherein the threaded mechanism is configured to facilitate operation in the gross-tensioning manner by allowing non-rotational axial movement of the knob with respect to the handle via transient disengagement of the threaded mechanism upon application of a non-rotational axial force to the knob.
